(19) United States
(12) Patent Application Publication
(10) Pub. No.: US 2008/0206804 A1
(43) Pub. Date: Aug. 28, 2008

(54) ARRANGEMENTS AND METHODS FOR MULTIDIMENSIONAL MULTIPLEXED LUMINESCENCE IMAGING AND DIAGNOSIS

(75) Inventors: Guillermo J. Tearney, Cambridge, MA (US); Brett E. Bouma, Quincy, MA (US); Leilei Peng, Quincy, MA (US); Tayyaba Hasan, Arlington, MA (US); Sarika Verma, Waltham, MA (US)

Correspondence Address: DORSEY & WHITNEY LLP;INTELLECTUAL PROPERTY DEPARTMENT, 250 PARK AVENUE, NEW YORK, NY, 10177, US

(73) Assignee: The General Hospital Corporation, Boston, MA (US)

(21) Appl. No.: 12/016,051

(22) Filed: Jan. 17, 2008

Related U.S. Application Data
(60) Provisional application No. 60/885,781, filed on Jan. 19, 2007.

(51) Int. Cl. C12Q 1/02; C12M 1/34; G01N 21/63; G01N 21/76

(52) U.S. Cl. 435/29; 435/288.7; 436/172; 422/82.05

(57) ABSTRACT

Exemplary embodiments of arrangements and methods providing information associated with a sample are described. For example, using such exemplary arrangements and methods, it is possible to receive an unpartitioned electro-magnetic radiation from the sample. Further, first data associated with first luminescent characteristics of at least one first molecule of the sample and second data associated with second luminescent characteristics of at least one second molecule of the sample can be obtained based on the unpartitioned electro-magnetic radiation. At least two of the photo-luminescent properties of the sample may be measured simultaneously as a function of the first and second data. Further, the information regarding the molecules of the sample may be determined as a function of the photo-luminescent properties.

### CROSS-REFERENCE TO RELATED APPLICATION(S)

This application is based upon and claims the benefit of priority from U.S. Patent Application Ser. No. 60/885,781, filed Jan. 19, 2007, the entire disclosure of which is incorporated herein by reference.

### STATEMENT REGARDING FEDERALLY SPONSORED RESEARCH

The invention was made with the U.S. Government support under Contract No. FA9550-04-1-0079 awarded by the Department of Defense. Thus, the U.S. Government has certain rights in the invention.

### FIELD OF THE INVENTION

The present invention relates to arrangements and methods for imaging and diagnosis with luminescent labels. In particular, the present invention is directed to arrangements and methods for multidimensional multiplexed luminescence imaging and diagnosis.

### BACKGROUND INFORMATION

Targeted imaging and diagnosis with luminescent (e.g. fluorescent, photoluminescent) labels has long been a standard tool for biology and medicine. For this technique, luminophores that have been modified to specifically bind to the target, such as certain tissues, cell molecules, proteins, or genes, are used as a tool for measuring or imaging the existence, location, and quantity of the target. It has become recognized that in living systems, many different molecular pathways operate in parallel for both normal and abnormal expression and function and in response to therapy. Dense multiplexing, defined as nearly simultaneous or parallel detection of at least more than 2 labels and extending to hundreds or thousands of labels, is therefore required in order to understand biology and disease on this systems level. In addition to imaging, multiplexing is highly useful for flow cytometry to characterize cell type and molecular expression from extracted samples.

At present, multiplexed fluorescence imaging and diagnosis is achieved by using fluorophore labels that can have distinguishable emission spectra, i.e. emission color or wavelength. Currently, the number of fluorophores that can be simultaneously detected is limited, due to technical challenges associated with filter bandwidths and detectors, as well as the bandwidths of fluorophore emission spectra, which are typically greater than 30 nm. Therefore, at best, only about 10 fluorophores can be used at any one time in an emission multiplexing system, and, a more practical limit ranges from 2-5 distinct labels. Furthermore, the choice of fluorophores that can be simultaneously excited, while exhibiting distinguishable emission spectra, is very limited.

Complete optical characterization of a luminophore includes temporal response, which is often characterized as decay lifetime, and spectral intensity. An additional mode of characterization is luminescent polarization anisotropy. These properties are two-dimensional functions of excitation wavelengths and emission wavelengths. Fluorophores that have overlapping spectral signature are potentially distinguishable by their lifetime. Measuring lifetime and excitation and emission spectral properties simultaneously can greatly improve the multiplexing density above and beyond the emission spectrum alone.

High throughput multiplexing requires luminescent labels with appropriately engineered optical signatures. Unlike most fluorophores, excitation and emission spectra of photoluminescent semiconductor nanocrystals, also called quantum dots, are determined mainly by the quantum confinement effect. Their lifetime, excitation and emission spectrum peak is particle size-dependent. Commercially available semiconductor nanocrystals have been demonstrated for emission multiplex imaging and detection. However, at present, nanocrystals labeled multiplex studies uses II-VI or III-V compound semiconductor crystals, which contain toxic elements such as Ce, Se, and As. Silicon nanocrystals are an alternative type of nanoparticle that also exhibit photoluminescence, which are biocompatible. Non-toxic silicon nanocrystals are highly advantageous, as they permit both live cell and in vivo imaging in animals and possibly even living human patients. Silicon nanocrystals also have unique long emission decay lifetimes that are on the order microseconds, which, as predicted by quantum confinement theory, are size dependent.

One of the objects of the present invention is to overcome the above-described deficiencies.

### SUMMARY OF EXEMPLARY EMBODIMENTS OF PRESENT INVENTION

In accordance with an exemplary embodiment of the present invention, a device and method can be provided that detects and distinguishes more than one distinct target using information related to at least one of photoluminescence lifetime, excitation, emission, and anisotropy characteristics. The exemplary device includes a spectroscopic instrument that can simultaneously measure at least one of the fluorescence, phosphorescence, photoluminescence, and polarization anisotropy properties, which include the temporal property as a function of excitation and/or emission wavelength, spectral intensities as a function of excitation and/or emission wavelength. In an exemplary embodiment, the use of silicon nanocrystals can be such that these crystals are stabilized via a surface modification and functionalized via attachment of an antibody that specifically binds to a distinct biological target.

Thus, according to certain exemplary embodiments of the present invention, arrangements and methods which can facilitate information associated with a sample may be provided. For example, using such exemplary arrangements and methods, it is possible to receive an unpartitioned electro-magnetic radiation from the sample. Further, first data associated with first luminescent characteristics of at least one first molecule of the sample and second data associated with second luminescent characteristics of at least one second molecule of the sample can be obtained based on the unpartitioned electro-magnetic radiation. At least two of the photo-luminescent properties of the sample may be measured simultaneously as a function of the first and second data. Further, the information regarding the molecules of the sample may be determined as a function of the photo-luminescent properties.

According to another exemplary embodiment of the present invention, is also possible to obtain third data associated with third luminescent characteristics of at least one third molecule of the sample. At least three of the photo-luminescent properties of the sample can then be measured simultaneously as a function of the first, second and third data. The photo-luminescent properties may include a lifetime, an excitation wavelength, an emission wavelength, and/or an anisotropy. The association between (i) the first data and the first luminescent characteristics and/or (ii) the second data and the second luminescent characteristics may be intrinsic and/or extrinsic.

According to yet another exemplary embodiment of the present invention, when the association is extrinsic, the first luminescent characteristics and/or the second luminescent characteristics may be chemically modifiable independently from one another. The information may be determined by differentiating between the first and second molecules as a function of the first luminescent characteristics and/or the second luminescent characteristics, e.g., when the first luminescent characteristics and the second luminescent characteristics are different from one another. The extrinsic association can include (i) a chemical modification and/or (ii) a genetic transcription of at least one of the first and/or second molecule(s).

According to a further exemplary embodiment of the present invention, the sample can includes an anatomical structure, a cell and/or a biological molecule. In addition, the information may be determined by ascertaining a first concentration of the first molecule and a second concentration the second molecule using the photo-luminescent properties. The information can also be determined by ascertaining an association between the first molecule and the second molecule.

These and other objects, features and advantages of the present invention will become apparent upon reading the following detailed description of embodiments of the present invention.

### BRIEF DESCRIPTION OF THE DRAWINGS

Further objects, features and advantages of the invention will become apparent from the following detailed description taken in conjunction with the accompanying figures showing illustrative embodiments of the invention, in which:

FIG. 1 is an exemplary graph generated by a conventional emission multiplexing procedure;

FIG. 2 is an exemplary graph generated by an exemplary embodiment of a multidimensional multiplexing procedure by measuring L-EEM or I-EEM according to the present invention;

FIG. 3 is n exemplary graph generated by an exemplary embodiment of a multidimensional multiplexing procedure by measuring L-EEM and I-EEM according to the present invention;

FIG. 4 is an exemplary illustration of an exemplary embodiment of an experimental result on measuring I-EEM of a mixture of two fluorophores;

FIG. 5 is an illustration of an exemplary embodiment of a system which is capable of performing an exemplary embodiment of a multidimensional multiplexing procedure;

FIG. 6 is an exemplary graph of exemplary results for prediction of quantum confinement procedure according to the exemplary embodiments of the present invention, in which shorter excitation wavelengths probe nanocrystals have smaller sizes, and therefore lifetimes corresponding to shorter excitation wavelengths can be smaller; and

FIG. 7 is an illustration of an exemplary structure of a functionized silicon nanocrystal.

Throughout the figures, the same reference numerals and characters, unless otherwise stated, are used to denote like features, elements, components or portions of the illustrated embodiments. Moreover, while the subject invention will now be described in detail with reference to the figures, it is done so in connection with the illustrative embodiments. It is intended that changes and modifications can be made to the described embodiments without departing from the true scope and spirit of the subject invention.

### DETAILED DESCRIPTION OF EXEMPLARY EMBODIMENTS

Properties of excited luminescence can be fully described by two two-dimensional function, lifetime excitation emission matrix (L-EEM) τ(kₑₓ,kₑₘ) and intensity excitation matrix (I-EEM) EEM (kₑₓ,kₑₘ). The emission spectrum of a fluorophore under an illumination source is the projection of I-EEM on the emission axis

S_{M}(σ₂)=∫S₀(σ₁)IEEM(σ₁,σ₂)dσ₂ (1)

where S₀(σ₁) is the illumination source spectrum. The excitation spectrum of the fluorophore is the projection of I-EEM on the excitation axis

S_{X}( )=∫q(σ₂)IEEM(σ₁,σ₂)dσ₂ (2)

where q(σ₂) is the spectral response of the detector, which could contain a spectrometer or narrowband filter. Properties of a fluorophore can be visualized as an object in a multi-dimensional space, whose dimensions are lifetime, spectral intensity, excitation wave vector and emission wave vector. Anisotropy may be incorporated as one or more additional dimensions. Measurement results can be represented as a projection of the object on the measured axis. Fluorophores are distinguishable as long as their projections do not overlap.

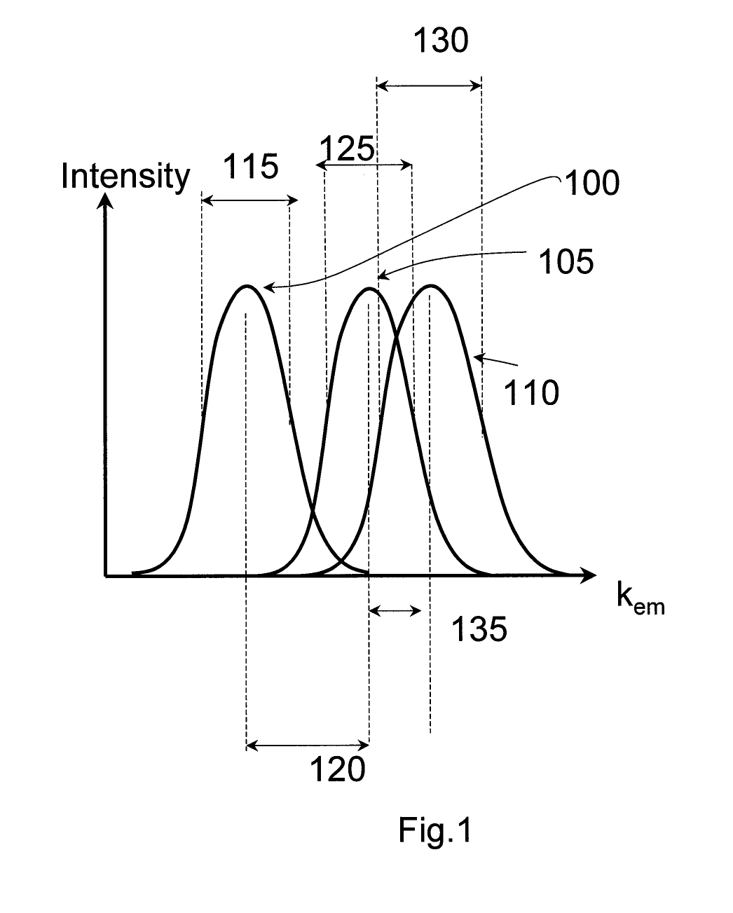

FIG. 1 depicts an example of prior art of emission multiplexing. As a rule of thumb, emission spectra of multiple fluorophore are distinguishable if half-width of their emission spectra is smaller than emission peak separations. 100, 105 and 110 are emission spectra of three different fluorophores. 100 and 105 can be distinguished by emission spectra because their emission peak separation 120 is lager than the average of their peak half width 115 and 125. 105 and 110 cannot be distinguished because peak separation 135 is smaller than the average of peak width 125 and 130.

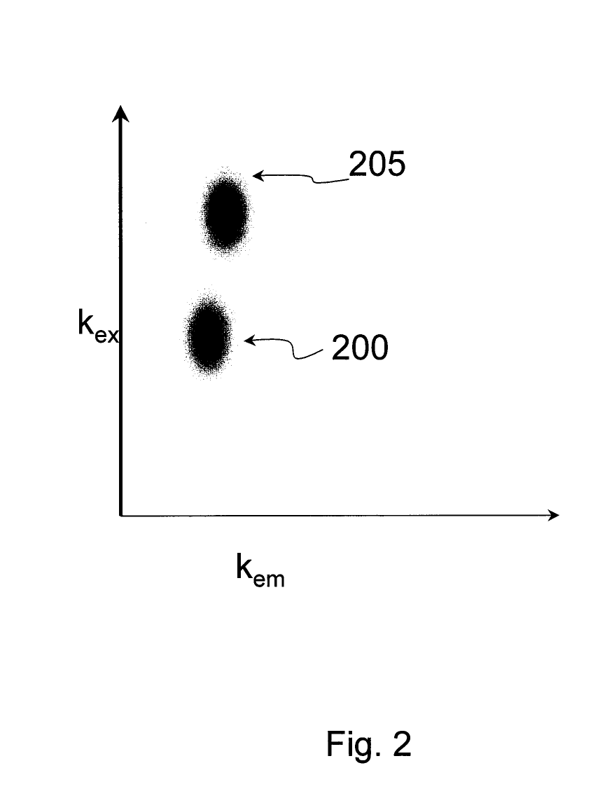

FIG. 2 depicts multidimensional multiplexing by measuring L-EEM (or I-EEM). L-EEM or I-EEM of two fluorophores 200 and 205 (grey scale map in the figure) are clearly separated in the excitation-emission two-dimensional space, although projections of L-EEM (or I-EEM) on the emission axis overlap.

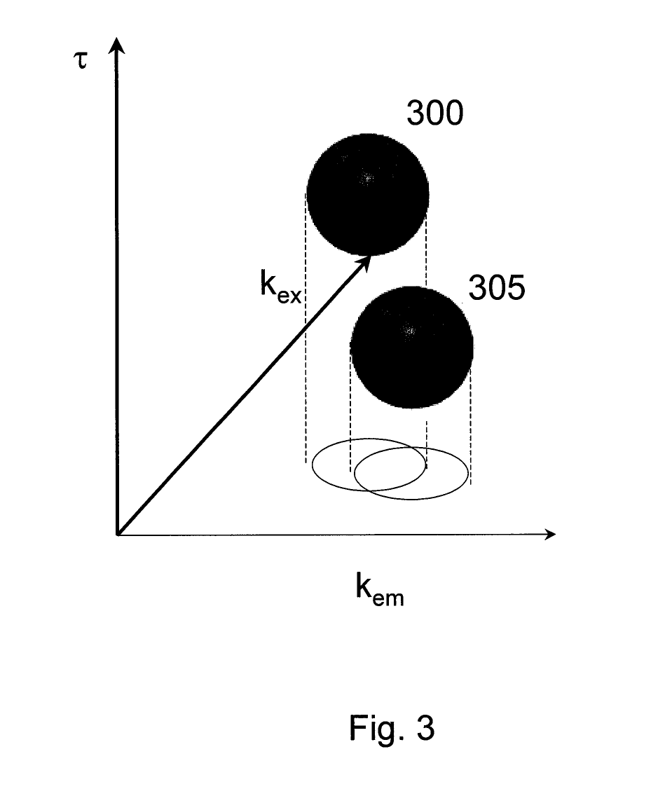

FIG. 3 depicts multiplexing by measuring L-EEM and I-EEM. Two fluorophores 300 and 305 (visualized as gray spheres) are clearly separated although they have overlap I-EEM (circular projection on kₑₓ-kₑₘ plane), however different lifetime (τ axis).

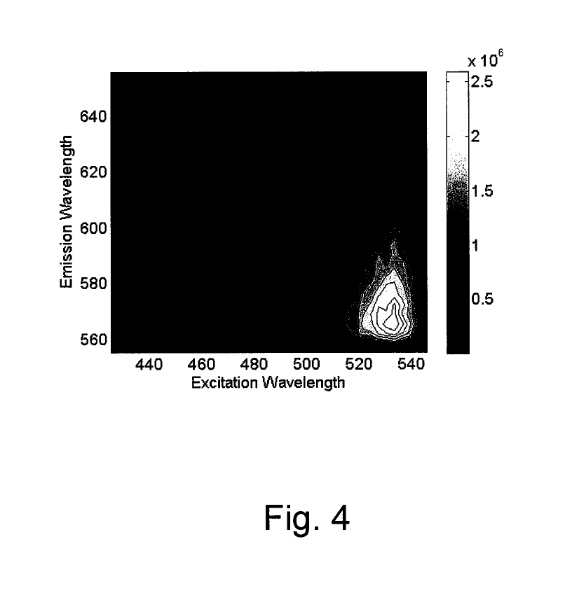

FIG. 4 is an experimental result on measuring I-EEM of a mixture of two fluorophores, Rhodamine 6G and Tris(2,2′-Bipyridyl) Ruthenium(II). Despite their overlapping emission and excitation spectra, these two fluorophores can be clearly distinguished by the I-EEM. Intensities peak at excitation 460 mm emission 610 nm are from Tris(2,2′-Bipyridyl) Ruthenium(II). Intensities peak at excitation 525 nm emission 560 nm are from Rhodamine 6G.

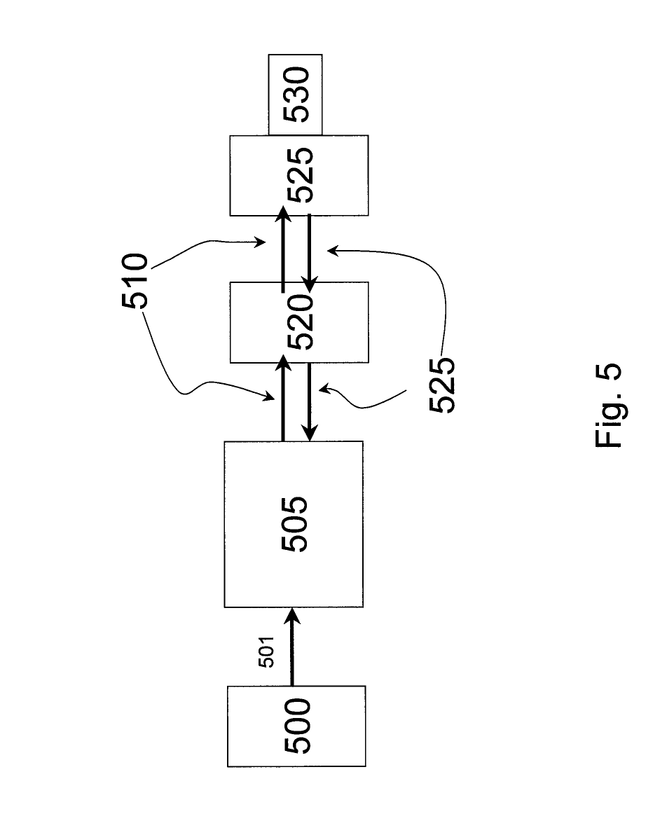

To achieve multidimensional luminescence multiplexing, exemplary devices that can simultaneously measurement lifetime, excitation, emission, and anisotropy of fluorophores (see, e.g., U.S. Patent Application No. 60/760,085 filed Jan. 19, 2006) can be used. For example, FIG. 5 shows an exemplary embodiment of multidimensional multiplexing device. The exemplary instrument may contain a light source (500), an optical instrument that performs multidimensional measurement (505). The instrument first performs spectral encoding on the light from 500 (501). Light 501 is sent into optical instrument 505. Spectrally and/or frequency encoded light 510 is focused onto the sample (515) by an objective (520). 520 collects the fluorescence emission (525) and send it back to 505 for detection. Imaging can be accomplished by either moving the sample with a translation stage (530), by scanning the focus of the objective lens, or by scanning the illuminating beam. In another embodiment, the detector may an imaging device such as a CCD or CMOS or ICCD camera coupled. The detector may be coupled to a spectrometer device or alternatively an interferometer for Fourier transform spectral detection.

An exemplary embodiment of the SLEE instrument according to the present invention is capable of determining and/or detecting, e.g., lifetime, excitation, emission, and anisotropy data of samples. Based on the data, various concentrations of multiple fluorescent targets in the samples can be recovered by the exemplary nonlinear unmixing method/procedure with prior knowledge regarding fluorescence characteristics of fluorescent targets. An exemplary multiplexing fluorescent image can be mathematically provided as:

$$M ( x , y , z , {σ}_{1} , {σ}_{2} , τ ) = {\sum }_{i} {C}_{i} ( x , y , z ) {IEEM}_{i} ( {σ}_{1} , {σ}_{2} ) δ ( τ - {τ}_{i} ) \qquad ( 3 )$$

where Cᵢ(x,y,z) can be the concentration distribution of the n^{th} targets, IEEMᵢ may be its steady state I-EEM of an unit concentration, and τᵢ may be its lifetime (assuming lifetime is a constant for a pure fluorophore). IEEMᵢ and τᵢ can be obtained by measuring pure fluorophores. Using such prior knowledge, a recovery of Cᵢ(x,y,z) can be considered as a non-linear unmixing problem, where the mixing function may follow the multi-exponential decay model.

A SFLEE instrument can measure raw data given by:

$${P}_{M} ( {σ}_{1} , {σ}_{2} ) = δ ( {σ}_{1} ) δ ( {σ}_{2} ) \int \int IEEM ( {σ}_{1} , {σ}_{2} ) {S}_{0} ( {σ}_{1} ) d {σ}_{1} d {σ}_{2} \\ + δ ( {σ}_{2} ) . \int mIEEM ( {σ}_{1} , {σ}_{2} ) {S}_{0} ( {σ}_{1} ) exp ( i φ ) d {σ}_{2} \\ + δ ( {σ}_{1} ) \int IEEM ( {σ}_{1} , {σ}_{2} ) {S}_{0} ( {σ}_{1} ) d {σ}_{1} \\ + mIEEM ( {σ}_{1} , {σ}_{2} ) {S}_{0} ( {σ}_{1} ) exp ( i φ ) \qquad ( 4 )$$

where δ(σ) is defined by

$$δ ( σ ) = \{ \begin{matrix}1 , σ = 0 \\ 0 , σ \neq 0\end{matrix} \qquad ( 5 )$$

Equation (4) herein above includes four terms. For example, the first term is the total steady state emission power; the second term is the one-dimensional excitation spectrum S_{X}(σ₁); the third term is the one-dimensional emission spectrum S_{M}(σ₂), and the last term is the raw EEM data, which can contain information about both the steady state I-EEM and the L-EEM

EEM_{Raw}=mIEEM(σ₁,σ₂)S₀(σ₁)exp(iφ) (6)

where m can be defined by:

m=(N²+D²)^{1/2} (7)

and N and D may be defined by:

$$\{ \begin{matrix}N = {\sum }_{i} \frac{4 π {C}_{i} ( x , y , z , ) IEEM ( {σ}_{1} , {σ}_{2} ) {σ}_{1} {v}_{1} {τ}_{i}}{( 1 + 16 {π}^{2} {σ}_{1}^{2} {v}_{1}^{2} {τ}_{i}^{2} )} / {\sum }_{i} {C}_{i} ( x , y , z ) IEEM ( {σ}_{1} , {σ}_{2} ) \\ D = {\sum }_{i} \frac{{C}_{i} ( x , y , z ) IEEM ( {σ}_{1} , {σ}_{2} )}{( 1 + 16 {π}^{2} {σ}_{1}^{2} {v}_{1}^{2} {τ}_{i}^{2} )} / {\sum }_{i} {C}_{i} ( x , y , z ) IEEM ( {σ}_{1} , {σ}_{2} )\end{matrix} \qquad ( 8 )$$

in which, e.g., only an exemplary quantity Cᵢ(x,y,z), highlighted in the equation above, may be unknown. For example, Cᵢ(x,y,z) can be independent of excitation emission wavenumbers (σ₁, σ₂). Thus, the concentration recovery can be considered as a global analysis problem with respect to the concentrations.

An exemplary expression of each targets can be determined simultaneously by, e.g., least square fitting on Cᵢ(x,y,z) with Equation 6 provided herein. Exemplary maps of locations and concentrations of each of the targets can be formed by repeating the least square fitting one each image point. Alternative additional linear combinations of data or statistical exemplary methods and/or procedures including partial least squares, principle component analysis, neural nets, or genetic procedures may be utilized to parse the multidimensional luminescence characteristic space to discriminate different targets. Alternatively or in addition, a clustering statistical systems and procedures including but not limited to Euclidean, Normalized, or Malahanobis distance, and classification methods, pattern recognition, and/or supervised learning may be utilized to discriminate different targets in the multidimensional luminescence characteristic space.

In addition, the association between multiple targets can be measured by, e.g., a Fluorescence Energy Transfer Effect, which can occur when two fluorescent targets (Donor and Acceptor) can have a distance within tens of nanometers. The IEEM of FRET signals may be provided as follows:

IEEM_{FRET}(σ₁,σ₂)=S_{X-Donor}(σ₁)S_{M-Acceptor}(σ₂) (9)

where S_{X-Donor}(σ₁) can be the excitation spectrum of the donor, and S_{M-Acceptor}(σ₂) may be the emission spectrum of the acceptor.

A fluorophore “palette” with tens to hundreds of unique L-EEM and I-EEM can be built for densely multiplexed imaging. Every fluorophore in the palette may be assigned to individual markers. For example, EEM's of labeled probes of uniform concentration can be measured with SFLEE prior to the exemplary imaging application. Such EEM's can be used in concentration recoveries in the exemplary imaging reconstruction. A list of roughly tens of fluorophores have been widely used in fluorescence imaging procedures. Such list provides enough information and materials for imaging biochemical markers in most biomedical applications, but likely not enough for certain applications where a long list of markers have been identified, such as, e.g., gene profiling. Further labeling strategies can be provided for these applications. One exemplary strategy that has been used generally utilizes combinations of fluorophores with FRET effects. In such exemplary procedures, probes labeled with a FRET pair may have a new I-EEM that is not the linear combination of the donor and the acceptor I-EEM.

Lifetime changes caused by FRET are also known. For example, exemplary probes labeled with a FRET pair can have unique L-EEM and I-EEM, which can be generated by controlling the FRET efficiency, for example by site-selective labeling during oligonucleotide synthesis. With one exemplary FRET pair combination, tens of genetic probes with distinguishable EEMs can be produced.

Another exemplary strategy/procedure can include the implementation of silicon quantum dots. Both theory calculation and experiment have demonstrated that silicon nanocrystals smaller that 5 nm in diameter generally emit luminescence under UV or blue illumination. Theory calculations and experimental observations also indicate that while the luminescence is generated by the quantum confinement effect in the nanocrystal structure, surface electron states also likely have an effect. Silicon nanocrystals generally have rich variations in both lifetime and spectral intensities that can be maneuverable by different core size/surface coating combinations.

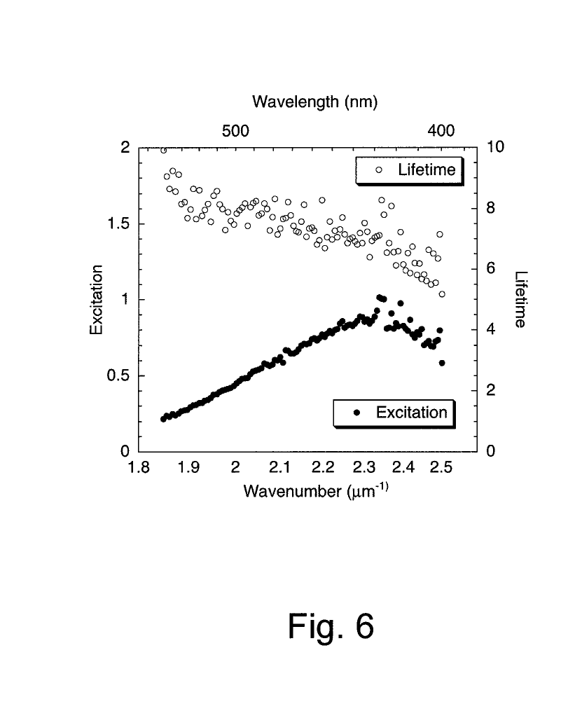

One exemplary prediction of quantum confinement theory can be, when the size of the nanocrystal decreases, excitation and emission spectra shift towards shorter wavelength, and emission lifetime decreases. FIG. 6 is the experiment result of the lifetime of porous silicon as a function of excitation wavelength, measured by the SFLEE device. Porous silicon is a material that contains numerous silicon nanocrystals at different sizes. Under the prediction of quantum confinement theory, shorter excitation wavelengths probe nanocrystals with smaller sizes, and therefore lifetimes corresponding to shorter excitation wavelengths should be smaller. The results shown FIG. 6 validate this prediction.

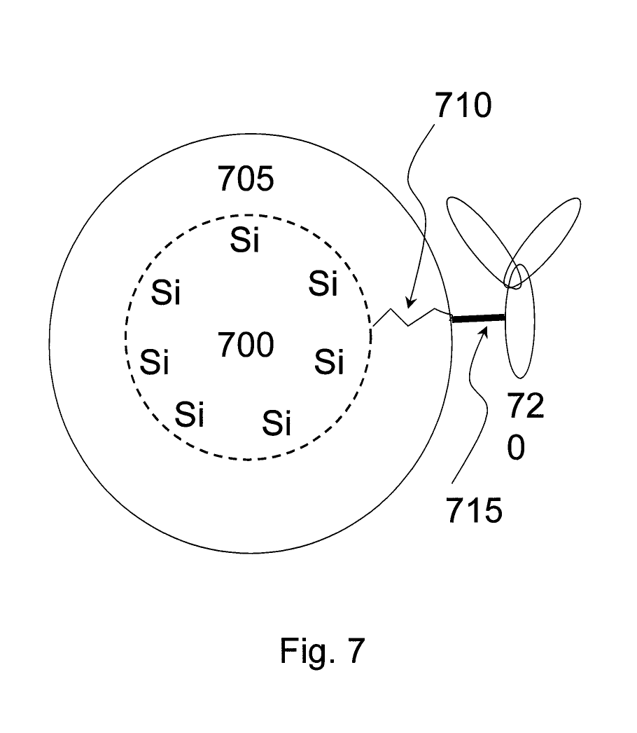

The purpose of coating is first preventing silicon nanocrystals from oxidization, second, providing function groups that can be further link to specific targets. FIG. 7 depicts a prior art of the structure of a functionized silicon nanocrystal. The crystal has a silicon core (700). A layer of organic coating (705) covalently bonds to the surface silicon atoms in the core via C—Si or Si—O—Si bonds. The organic coating molecule consists of a carbon chain (710) and a reactive group (715) on the outside. The reactive group (715) is linked with a biological molecule (720), for example, an antibody. The biological molecule (720) has specific binding to the target that the nanocrystal is designed to detect. Silicon nanocrystals with different L-EEM and I-EEM are engineered by changing the core size and the organic coating combination.

The foregoing merely illustrates the principles of the invention. Various modifications and alterations to the described embodiments will be apparent to those skilled in the art in view of the teachings herein. Indeed, the arrangements, systems and methods according to the exemplary embodiments of the present invention can be used with any OCT system, OFDI system, spectral domain OCT (SD-OCT) system or other imaging systems, and for example with those described in International Patent Application PCT/US2004/029148, filed Sep. 8, 2004, U.S. patent application Ser. No. 11/266,779, filed Nov. 2, 2005, and U.S. patent application Ser. No. 10/501,276, filed Jul. 9, 2004, the disclosures of which are incorporated by reference herein in their entireties. It will thus be appreciated that those skilled in the art will be able to devise numerous systems, arrangements and methods which, although not explicitly shown or described herein, embody the principles of the invention and are thus within the spirit and scope of the present invention. In addition, to the extent that the prior art knowledge has not been explicitly incorporated by reference herein above, it is explicitly being incorporated herein in its entirety. All publications referenced herein above are incorporated herein by reference in their entireties.

What is claimed is:

1. An apparatus for providing information associated with a sample, comprising:
a first arrangement which is configured to receive an unpartitioned electro-magnetic radiation from the sample, obtain first data associated with first luminescent characteristics of at least one first molecule of the sample and second data associated with second luminescent characteristics of at least one second molecule of the sample based on the unpartitioned electro-magnetic radiation, and measure at least two of the photo-luminescent properties of the sample simultaneously as a function of the first and second data; and
a second arrangement which is configured to determine the information regarding the molecules of the sample as a function of the photo-luminescent properties.

2. The arrangement according to claim 1, wherein the first arrangement is further configured to obtain third data associated with third luminescent characteristics of at least one third molecule of the sample, and measure at least three of the photo-luminescent properties of the sample simultaneously as a function of the first, second and third data.

3. The arrangement according to claim 1, wherein the photo-luminescent properties include at least one a lifetime, an excitation wavelength, an emission wavelength, or an anisotropy.

4. The arrangement according to claim 1, wherein the association between at least one of (i) the first data and the first luminescent characteristics or (ii) the second data and the second luminescent characteristics is intrinsic.

5. The arrangement according to claim 1, wherein the association between at least one of (i) the first data and the first luminescent characteristics or (ii) the second data and the second luminescent characteristics is extrinsic.

6. The arrangement according to claim 5, wherein at least one of the first luminescent characteristics or the second luminescent characteristics which are extrinsic are chemically modifiable independently from one another.

7. The arrangement according to claim 1, wherein the second arrangement determines the information by differentiating between the first and second molecules as a function of at least one of the first luminescent characteristics or the second luminescent characteristics, when the first luminescent characteristics and the second luminescent characteristics are different from one another.

8. The arrangement according to claim 5, wherein the extrinsic association includes at least one of (i) a chemical modification or (ii) a genetic transcription of the at least one of the first molecule or the second molecule.

9. The arrangement according to claim 1, wherein the sample includes an anatomical structure, a cell or a biological molecule.

10. The arrangement according to claim 1, wherein the second arrangement is further configured to determine the information by determining a first concentration of the at least one first molecule and a second concentration the at least one second molecule using the photo-luminescent properties.

11. The arrangement according to claim 1, wherein the second arrangement is further configured to determine the information by determining an association between the at least one first molecule and the at least one second molecule.

12. A method for providing information associated with a sample, comprising:
receiving an unpartitioned electro-magnetic radiation from the sample;
obtaining first data associated with first luminescent characteristics of at least one first molecule of the sample and second data associated with second luminescent characteristics of at least one second molecule of the sample based on the unpartitioned electro-magnetic radiation;
measuring at least two of the photo-luminescent properties of the sample simultaneously as a function of the first and second data; and
determining the information regarding the molecules of the sample as a function of the photo-luminescent properties.

13. The method according to claim 12, further comprising:
obtaining third data associated with third luminescent characteristics of at least one third molecule of the sample; and
measuring at least three of the photo-luminescent properties of the sample simultaneously as a function of the first, second and third data.

14. The method according to claim 12, wherein the photo-luminescent properties include at least one a lifetime, an excitation wavelength, an emission wavelength, or an anisotropy.

15. The method according to claim 12, wherein the association between at least one of (i) the first data and the first luminescent characteristics or (ii) the second data and the second luminescent characteristics is intrinsic.

16. The method according to claim 12, wherein the association between at least one of (i) the first data and the first luminescent characteristics or (ii) the second data and the second luminescent characteristics is extrinsic.

17. The method according to claim 16, wherein at least one of the first luminescent characteristics or the second luminescent characteristics which are extrinsic are chemically modifiable independently from one another.

18. The method according to claim 12, wherein the information is determined by differentiating between the first and second molecules as a function of at least one of the first luminescent characteristics or the second luminescent characteristics, when the first luminescent characteristics and the second luminescent characteristics are different from one another.

19. The method according to claim 16, wherein the extrinsic association includes at least one of (i) a chemical modification or (ii) a genetic transcription of the at least one of the first molecule or the second molecule.

20. The method according to claim 12, wherein the sample includes an anatomical structure, a cell or a biological molecule.

21. The method according to claim 12, wherein the information is determined by ascertaining a first concentration of the at least one first molecule and a second concentration the at least one second molecule using the photo-luminescent properties.

22. The method according to claim 12, wherein the information is determined by ascertaining an association between the at least one first molecule and the at least one second molecule.
